(19) United States
(12) Patent Application Publication
(10) Pub. No.: US 2013/0303458 A1
(43) Pub. Date: Nov. 14, 2013

(54) SUBSTANCES AND METHODS FOR THE TREATMENT OF B CELL MEDIATED MULTIPLE SCLEROSIS

(71) Applicant: SUPREMOL GMBH, Martinsried/ Muenchen (DE)

(72) Inventors: Peter BUCKEL, Bernried (DE); Uwe JACOB, Munich (DE)

(21) Appl. No.: 13/912,859

(22) Filed: Jun. 7, 2013

Related U.S. Application Data
(62) Division of application No. 13/062,115, filed on Jun. 3, 2011, now abandoned.

(30) Foreign Application Priority Data
Sep. 5, 2008 (EP) 08163800.9

(51) Int. Cl. C07K 14/735

(52) U.S. Cl. CPC C07K 14/70535
USPC 514/17.9

(57) ABSTRACT

The invention relates to the Fcγ receptor (Fc-gamma receptor) for use in treating multiple sclerosis, wherein the multiple sclerosis is a B cell mediated form of multiple sclerosis and/or an autoantibody driven form of multiple sclerosis. The invention relates to pharmaceutical compositions containing the Fcγ receptor (Fc-gamma receptor) for use in treating multiple sclerosis, wherein the multiple sclerosis is a B cell mediated form of multiple sclerosis and/or an autoantibody driven form of multiple sclerosis.

The present application is a divisional of co-pending U.S. patent application Ser. No. 13/062,115, filed as a national phase application under 35 U.S.C. §371 of International Application No. PCT/EP2009/061359, filed Sep. 2, 2009 which claims priority to European Application No. 08163800.9 filed on Sep. 5, 2008. The entire contents of each are herein incorporated by reference.

### FIELD OF THE INVENTION

The invention is in the field of biotechnology and therapeutics. The invention relates to the Fcγ receptor (Fc-gamma receptor) for use in treating multiple sclerosis, wherein the multiple sclerosis is a B cell mediated form of multiple sclerosis and/or an autoantibody driven form of multiple sclerosis.

### BACKGROUND OF THE INVENTION

Current hypotheses favour the concept that T cells play a pivotal role in the pathogenesis of multiple sclerosis (MS), which was initially based upon the observation that T cells are the predominant lymphocyte class present in MS lesions (Windhagen, et al., Cytokine, secretion of myelin basic protein reactive T cells in patients with multiple sclerosis. Journal of Neuroimmunology, 91:1-9, 1998; Hafler, D. A., et al., Oral administration of myelin induces antigen-specific TGF-beta 1 secreting cells in patients with multiple sclerosis. Annals of the New York Academy of Science, 835:120-131, 1997; Lovett-Racke, A. E., et al., Decreased dependence of myelin basic protein-reactive T cells on CD28-mediated costimulation in multiple sclerosis patients, Journal of Clinical Investigation, 101:725-730, 1998) This observation continues to be a cardinal hallmark of the disease, and is supported by a number of observations. For example, active CD4+ T helper cells bearing anti-myelin T Cell Receptors (TCRs) are present in the cerospinal fluid (CSF) of patients with MS. In addition, elevated levels of Th1-like cytokines have been detected in the CSF of patients with MS and have been correlated with worsening of the disease in some cases (Calabresi et al, Cytokine expression in cells derived from CSF of multiple sclerosis patients. Journal of Neuroimmunology, 89:198-205, 1998).

There has, however, also been evidence that B cells may be involved in the development and perpetuation of MS including:

(1) elevated immunoglobulin levels in the CSF of MS patients (Link, H., et al., Immunoglobulins in multiple sclerosis and infections of the nervous system, Archives of Neurology, 25:326-344, 1971; Link, H., et al., Immunoglobulin class and light chain type of oligoclonal bands in CSF in multiple sclerosis determined by agarose gel electrophoresis and immunofixation. Ann Neurol, 6(2):107-110, 1979; Perez, L., et al., B cells capable of spontaneous IgG secretion in cerebrospinal fluid from patients with multiple sclerosis: dependancy on local IL-6 production. Clinical Experimental Immunology, 101:449-452, 1995),

(2) oligoclonal banding in the CSF of MS patients (Link, H., et al., Immunoglobulin class and light chain type of oligoclonal bands in CSF in multiple sclerosis determined by agarose gel electrophoresis and immunofixation. Ann Neurol, 6(2):107-110, 1979),

(3) skewing of the κ:λ ratio in the CSF of MS patients (Hauser, S. L., et al., Clonally restricted B cells in peripheral blood of multiple sclerosis patients: kappa/lambda staining patterns. Annals of Neurology, 11:408-412, 1982),

(4) the presence of anti-myelin antibodies in the CSF of MS patients (Sun, J. H., et al, B cell responses to myelin-oligodendrocyte glycoprotein in multiple sclerosis. Journal of Immunology, 146:1490-1495, 1991) and

(5) the demonstration that antibodies from the CSF of MS patients may contribute to the overall extent of tissue injury in these patients (Lassmann, H., et al., Experimental allergic encephalomyelitis: the balance between encephalitogenic T lymphocytes and demyelinating antibodies determines size and structure of demyelinated lesions. Acta Neuropathology, 75:566-576, 1988).

A number of publications demonstrate such B cell mediation: Bourquin, et al., The journal of Immunology, 2003, 171: 455-461; Stromnes et al. Nature Protocols, Vol. 1, NO: 4, 2006: 1810-1818; Stromnes et al Nature Protocols, Vol. 1, NO: 4, 2006: 1952-19160; Oliver, et al., The Journal of Immunology, 2003, 171: 462-468;

The basic role of B cells in the immune system is to mediate Humoral Immune responses. That is, to secrete proteins called antibodies (or immunoglobulins) that bind to foreign bodies and mark them for elimination from the body by other immune cells such as NK cells and macrophages.

Intravenous immunoglobulin (IVIG) has been demonstrated in multiple clinical trials reported in the medical literature to have an impact on two important considerations in relapsing-remitting Multiple Sclerosis. IVIG reduces the frequency of the acute exacerbation and it reduces the intensity and duration of the acute exacerbation. Seemingly, a subset of patients has a prominent B cell involvement in multiple sclerosis. Hence, it would be advantageous to have substances and methods for the treatment of such B cell mediated multiple sclerosis patients.

IVIG represents pooled human immunoglobulin from many donors. The exact mechanism by which IVIG ameliorates autoantibody mediated diseases is unknown. However, it could be shown that a) the efficacy of IVIG is dependant on Fc-receptor expression b) the ratio of autoantibodies to normal immunoglobulin (IVIG) is shifted, which leads to enhanced degradation of autoantibodies and c) the IVIG contains the complete human antibody and anti-idiotype antibody network (antibodies against antibodies) which leads to the neutralization of autoantibodies.”

### SUMMARY OF THE INVENTION

The inventors have astonishingly found that the current hypothesis that T cells play a pivotal role in the pathogenesis of multiple sclerosis (MS) is at least not entirely correct. The inventors addressed the B cell mediated form of multiple sclerosis and are, astonishingly able to provide for a special treatment.

The invention relates to the Fcγ receptor (Fc-gamma receptor) for use in treating multiple sclerosis, wherein the multiple sclerosis is a B cell mediated form of multiple sclerosis and/or an autoantibody driven form of multiple sclerosis. The invention relates to pharmaceutical compositions containing the Fcγ receptor (Fc-gamma receptor) for use in treating multiple sclerosis, wherein the multiple sclerosis is a B cell mediated form of multiple sclerosis and/or an autoantibody driven form of multiple sclerosis.

Fc receptors (FcRs) play a key role in defending the human organism against infections. After pathogens have gained access to the blood circulation they are opsonized by immunoglobulins (Igs). The resulting immunocomplexes bind due to their multivalency with high avidity to FcR bearing cells leading to clustering of the FcRs, which triggers several effector functions (Metzger, H., 1992A). These include, depending on the expressed FcR type and associated proteins, endocytosis with subsequent neutralization of the pathogens and antigen presentation, antibody-dependent cellular cytotoxity (ADCC), secretion of mediators or the regulation of antibody production (Fridman, et al., 1992; van de Winkel and Capel, 1993).

Specific FcRs exist for all Ig classes, the ones for IgG being the most abundant with the widest diversity. Together with the high affinity receptor for IgE (FcεRIa), FcγRI (CD64), FcγRII (CD32) and FcγRIIIa (CD16) occur as type I transmembrane proteins or in soluble forms (sFcRs) but also a glycosylphosphatidylinositol anchored form of the FcγRIII (FcγRIIIb) exists. Furthermore, FcγRs occur in various isoforms (FcγRIa, b1, b2, c; FcγRIIa1-2, b1-3, c) and alleles (FcγRIIa1-HR, -LR; FcγRIIIb-NA1, —NA2) (van de Winkel and Capel, 1993). In contrast to the overall homologous extracellular parts, the membrane spanning and the cytoplasmic domains differ. They may be deleted entirely or be of a size of 8 kDa. They may contain either a 26 amino acid immunoreceptor tyrosine-based activation motif (ITAM) as in FcγRIIa or a respective 13 amino acid inhibitory motif (ITIM) in FcγRIIb involved in signal transduction (Amigorena, et al., 1992).

Herein, EAE is experimental autoimmune encephalomyelitis.

### DETAILED DESCRIPTION OF THE INVENTION

The invention relates to the Fcγ receptor (Fc-gamma receptor) for use in treating multiple sclerosis, wherein the multiple sclerosis is a B cell mediated form of multiple sclerosis and/or an autoantibody driven form of multiple sclerosis.

The B cell mediation of the multiple sclerosis and/or autoantibody driven form of multiple sclerosis is characterized by one or more of the following features, (a) the multiple sclerosis is ameliorated if the patient undergoes intravenous immunoglobulin (IVIG) treatment and/or, (b) the multiple sclerosis is ameliorated if the patient undergoes anti-CD20 antibody treatment and/or, (c) the multiple sclerosis is ameliorated if the patient undergoes plasmapheresis and/or, (d) the multiple sclerosis is ameliorated if the patient undergoes immunoadsorption (e) the presence of autoantibodies against the antigen myelin oligodendrocyte glycoprotein (MOG) and/or (f) the presence of autoantibodies against the antigen myelin basic protein (MBP) and/or (g) the presence of autoantibodies against aquaporin 4.

Devic's disease is similar to MS in that the body's immune system attacks the myelin surrounding nerve cells. Unlike standard MS, the attacks are not believed to be mediated by the immune system's T cells but rather by antibodies called NMO-IgG. These antibodies target a protein called aquaporin 4 in the cell membranes of astrocytes which acts as a channel for the transport of water across the cell membrane.

In a preferred embodiment the B cell mediation of the multiple sclerosis and/or autoantibody driven form of multiple sclerosis is determined prior to the use of the Fcγ receptor by means of one or more of the following tests, (a) determining whether the multiple sclerosis is ameliorated if the patient undergoes intravenous immunoglobulin (IVIG) treatment and/or, (b) the multiple sclerosis is ameliorated if the patient undergoes anti-CD20 antibody treatment and/or, (c) the multiple sclerosis is ameliorated if the patient undergoes plasmapheresis, (d) the multiple sclerosis is ameliorated if the patient undergoes immunoadsorption (e) determining whether autoantibodies against the antigen myelin oligodendrocyte glycoprotein (MOG) are present in the patient and/or, (f) determining whether autoantibodies against the antigen myelin basic protein (MBP) are present in the patient and/or (g) determining the presence of autoantibodies against aquaporin 4. Preferably a selection of two of the test are performed, more preferably a selection of three of tests are performed, more preferably a selection of four of the tests are performed.

One example of an anti-CD20 treatment is Rituximab. This is a monoclonal antibody. Sadly, it has severe side-effects which could be ameliorated if the present protein, i.e. polypeptides were used in place of Rituximab.

During plasmapheresis, blood is initially taken out of the body through a needle or previously implanted catheter. Plasma is then removed from the blood by a cell separator. Three procedures are commonly used to separate the plasma from the blood:

### Discontinuous Flow Centrifugation:

One venous catheter line is required. Typically, a 300 ml batch of blood is removed at a time and centrifuged to separate plasma from blood cells.

### Continuous Flow Centrifugation

Two venous lines are used. This method requires slightly less blood volume to be out of the body at any one time as it is able to continuously spin out plasma.

### Plasma Filtration

Two venous lines are used. The plasma is filtered using standard hemodialysis equipment. This continuous process requires less than 100 ml of blood to be outside the body at one time.

During immunoadsorption the blood of a patient is cleared from immunoglobulin by an extra corporal affinity chromatography column.

Each method has its advantages and disadvantages. After plasma separation, the blood cells are returned to the person undergoing treatment, while the plasma, which contains the antibodies, is first treated and then returned to the patient in traditional plasmapheresis. (In plasma exchange, the removed plasma is discarded and the patient receives replacement donor plasma or saline with added proteins.) Medication to keep the blood from clotting (an anticoagulant) is generally given to the patient during the procedure. Plasmapheresis is used as a therapy in particular diseases.

An important use of plasmapheresis is in the therapy of autoimmune disorders. However, the method is extremely strenuous for the patient.

WO^{o}2008/017363 discloses means for testing for B cell mediation. In particular it discloses means of detecting autoantibodies against MOG and aquaporin 4. WO^{o}2008/017363 is incorporated by reference.

In a preferred embodiment of the invention the FcR receptor is of human origin. The Fcγ receptor according to the invention is preferably selected from the group of, FcγRI (CD64), FcγRIIA (CD32), FcγRIIB1 (CD32), FcγRIIB2 (CD32), FcγRIIc (CD32), FcγRIIIA (CD16) and FcγRIIIB (CD16).

FcγRIIB1 (CD32) and FcγRIIB2 (CD32) are so called isoforms, i.e. the isoforms 1 and 2.

According to the present invention, the preparation of the soluble Fc receptors takes place in prokaryotic or eukaryotic cells. It may also take place in eukaryotic cells. If it takes place in prokaryotic cells (see EP-B1 1 135 486) insoluble inclusion bodies containing the recombinant protein form, thus facilitating purification by separation of the inclusion bodies from other cell components before renaturation of the proteins contained therein takes place. The renaturation of the FcRs according to the present invention which are contained in the inclusion bodies can principally take place according to known methods. The advantage of the preparation in prokaryotic cells, the production of inclusion bodies and the thus obtained recombinant soluble Fc receptors make it possible to obtain a very pure and, in particular, also very homogeneous FcR preparation. Also because of the absence of glycosylation the obtained product is of great homogeneity. However, in some cases glycosylation may be desired.

A host cell is genetically engineered with the polynucleotide or the vector encoding or carrying the FcR. The host cells that may be used for purposes of the invention include but are not limited to prokaryotic cells such as bacteria (for example, E. coli and B. subtilis), which can be transformed with, for example, recombinant bacteriophage DNA, plasmid DNA, or cosmid DNA expression vectors containing the polynucleotide molecules encoding the FcR; simple eukaryotic cells like yeast (for example, Saccharomyces and Pichia), which can be transformed with, for example, recombinant yeast expression vectors containing the polynucleotide molecule of the invention, i.e. the polynucleotide molecules encoding the FcR; insect cell systems like, for example, Sf9 of Hi5 cells, which can be infected with, for example, recombinant virus expression vectors (for example, baculovirus) containing the polynucleotide molecules of the invention; Xenopus oocytes, which can be injected with, for example, plasmids; plant cell systems, which can be infected with, for example, recombinant virus expression vectors (for example, cauliflower mosaic virus (CaMV) or tobacco mosaic virus (TMV)) or transformed with recombinant plasmid expression vectors (for example, Ti plasmid) containing a FcR or variant nucleotide sequence; or mammalian cell systems (for example, COS, CHO, BHK, HEK293, VERO, HeLa, MDCK, Wi38, Swiss 3T3 and NIH 3T3 cells), which can be transformed with recombinant expression constructs containing, for example, promoters derived, for example, from the genome of mammalian cells (for example, the metallothionein promoter) from mammalian viruses (for example, the adenovirus late promoter, CMV IE and the vaccinia virus 7.5K promoter) or from bacterial cells (for example, the tet-repressor binding is employed in the tet-on and tet-off systems). Also useful as host cells are primary or secondary cells obtained directly from a mammal and transfected with a plasmid vector or infected with a viral vector. Depending on the host cell and the respective vector used to introduce the polynucleotide of the invention the polynucleotide can integrate, for example, into the chromosome or the mitochondrial DNA or can be maintained extrachromosomally like, for example, episomally or can be only transiently comprised in the cells. In the sequence of FcγRIIb three potential N-glycosylation sites are found. All three sites are on the surface of the molecule and are accessible. They are located in the EIF loops (N61 and N142) of both domains and on strand E (N 135) of the C-terminal domain. FcRs isolated from mammalian cells are highly glycosylated. Since FcR is glycosylated in vivo it may be desirable to chose an expression system, which provides faithful glycosylation of the protein. Consequently, it is preferred to introduce the polynucleotides encoding the FcR of the present invention into higher eukaryotic cells, in particular into mammalian cells, e.g. COS, CHO, BHK, HEK293, VERO, HeLa, MDCK, Wi38, Swiss 3T3 or NIH 3T3 cells.

Preferably the Fcγ receptor according to the invention lacks the transmembrane domain and/or the signal peptide and is soluble. Soluble forms of Fc receptors (sFcR) such as FcγRIII mediate isotype-specific regulation of B cell growth and immunoglobulin production. In a murine model of myeloma, sFcR suppresses growth and immunoglobulin production of tumor cells (Muller, et al., 1985; Roman, et al., 1988; Teillaud, et al., 1990). Furthermore, sFcR binds to surface IgG on cultures of human IgG-secreting myeloma cells and effects suppression of tumor cell growth and IgG secretion. Prolonged exposure of these cells to sFcR results in tumor cell cytolysis (Hoover, et al., 1995).

The Fcγ receptor polypeptides can be any of those described above but with not more than ten (e.g., not more than: ten, nine, eight, seven, six, five, four, three, two, or one) conservative substitutions. Conservative substitutions are known in the art and typically include substitution of, e.g. one polar amino acid with another polar amino acid and one acidic amino acid with another acidic amino acid. Accordingly, conservative substitutions preferably include substitutions within the following groups of amino acids: glycine, alanine, valine, proline, isoleucine, and leucine (non polar, aliphatic side chain); aspartic acid and glutamic acid (negatively charged side chain); asparagine, glutamine, methionine, cysteine, serine and threonine (polar uncharged side chain); lysine, histidine and arginine; and phenylalanine, tryptophane and tyrosine (aromatic side chain); and lysine, arginine an histidine (positively charged side chain). It is well known in the art how to determine the effect of a given substitution, e.g. on pKI etc. All that is required of a polypeptide having one or more conservative substitutions is that it has at least 50% (e.g., at least: 55%; 60%; 65%, 70%; 75%; 80%; 85%; 90%; 95%; 98%; 99%; 99.5%; or 100% or more) of the activity of the unaltered Fcγ receptor according to the invention.

Both polypeptides and peptides can be produced by standard in vitro recombinant DNA techniques and in vivo transgenesis, using nucleotide sequences encoding the appropriate polypeptides or peptides. Methods well-known to those skilled in the art can be used to construct expression vectors containing relevant coding sequences and appropriate transcriptional/translational control signals. See, for example, the techniques described in Sambrook, et al., Molecular Cloning: A Laboratory Manual (2nd Ed.) [Cold Spring Harbor Laboratory, N.Y., 1989], and Ausubel, et al., Current Protocols in Molecular Biology [Green Publishing Associates and Wiley Interscience, N.Y., 1989].

Polypeptides and fragments of the invention, i.e. isolated polypeptides, also include those described above, but modified for in vivo use by the addition, at the amino- and/or carboxyl-terminal ends, of blocking agents to facilitate survival of the relevant polypeptide in vivo. This can be useful in those situations in which the peptide termini tend to be degraded by proteases prior to cellular up-take. Such blocking agents can include, without limitation, additional related or unrelated peptide sequences that can be attached to the amino and/or carboxyl terminal residues of the peptide to be administered. This can be done either chemically during the synthesis of the peptide or by recombinant DNA technology by methods familiar to artisans of average skill.

Alternatively, blocking agents such as pyroglutamic acid or other molecules known in the art can be attached to the amino and/or carboxyl terminal residues, or the amino group at the amino terminus or carboxyl group at the carboxyl terminus can be replaced with a different moiety. Likewise, the peptides can be covalently or noncovalently coupled to pharmaceutically acceptable “carrier” proteins prior to administration.

The term “isolated” polypeptide or peptide fragment as used herein refers to a polypeptide or a peptide fragment which either has no naturally-occurring counterpart or has been separated or purified from components which naturally accompany it, e.g., in tissues such as tongue, pancreas, liver, spleen, ovary, testis, muscle, joint tissue, neural tissue, gastrointestinal tissue or tumor tissue, or body fluids such as blood, serum, or urine. Typically, the polypeptide or peptide fragment is considered “isolated” when it is at least 70%, by dry weight, free from the proteins and other naturally-occurring organic molecules with which it is naturally associated. Preferably, a preparation of a polypeptide (or peptide fragment thereof) of the invention is at least 80%, more preferably at least 90%, and most preferably at least 99%, by dry weight, the polypeptide (or the peptide fragment thereof), respectively, of the invention. Thus, for example, a preparation of polypeptide x is at least 80%, more preferably at least 90%, and most preferably at least 99%, by dry weight, polypeptide x. Since a polypeptide that is chemically synthesized is, by its nature, separated from the components that naturally accompany it, the synthetic polypeptide is “isolated.”

An isolated polypeptide (or peptide fragment) of the invention can be obtained, for example, by extraction from a natural source (e.g., from tissues or bodily fluids); by expression of a recombinant nucleic acid encoding the polypeptide; or by chemical synthesis. A polypeptide that is produced in a cellular system different from the source from which it naturally originates is “isolated,” because it will necessarily be free of components which naturally accompany it. The degree of isolation or purity can be measured by any appropriate method, e.g., column chromatography, polyacrylamide gel electrophoresis, or HPLC analysis.

The Fcγ receptor according to the invention may be chemically modified (improved) by PEGylation and/or genetic engineering.

Known approaches involve the provision of additional glycosylation sites (see e.g. WO 91/05867, WO 94/09257 and WO 01/81405). Such modified analogs may have at least one additional N-linked and/or O-linked carbohydrate chain. Other attempts to improve the half life may involve the addition of polyethylene glycol residues (PEG) of varying length to the amino acid backbone (see e.g. WO 00/32772, WO 01/02017, WO 03/029291). One may modify the molecules with at least one N-linked and/or O-linked oligosaccharide which are further modified by oxidation, sulfation, these approaches can equally be employed to extend the half life of the variants of the present invention and accordingly in a preferred embodiment of the Fcγ receptor according to the invention the modification is selected from the group consisting of oxidation, sulfation, phosphorylation, addition of oligosaccharides or combinations thereof. If the addition of further N-linked or O-linked oligonucleotides is desired it is possible to introduce them by introducing additional glycosylation sites. It also preferred that the protein is affinity modulated.

In the practice of one aspect of the present invention, a pharmaceutical composition comprising the receptor of the invention may be administered to a mammal by any route which provides a sufficient level of activity. It can be administered systemically or locally. Such administration may be parenterally, transmucosally, e.g., orally, nasally, rectally, intravaginally, sub-lingually, submucosally or transdermally. Preferably, administration is parenteral, e.g. via intravenous or intraperitoneal injection, and also including, but is not limited to, intraarterial, intramuscular, intradermal and subcutaneous administration. If the pharmaceutical composition of the present invention is administered locally it can be injected directly into the organ or tissue to be treated. In cases of treating the nervous system this administration route includes, but is not limited to, the intracerebral, intraventricular, intracerebroventricular, intrathecal, intracistemal, intraspinal and/or peri-spinal routes of administration, which can employ intracranial and intravertebral needles, and catheters with or without pump devices.

The receptor may also by glycosylated.

Most preferably the receptor is selected from the group of FcγRIIA/B/C(CD32) and FcγRIIIA/B (CD16b). The invention also relates to isoforms thereof and isoforms of the FcRs claimed herein in general.

In a preferred embodiment of pharmaceutical composition comprises an FcR receptor polypeptide in a dosage unit form for treating multiple sclerosis, wherein the multiple sclerosis is a B cell mediated form of multiple sclerosis and/or an autoantibody driven form of multiple sclerosis, and the amount to administered to a patient in a single dose is between 1 and 20 mg/kg, preferably 2 and 10 mg/kg, more preferably between 25 and 5 mg/kg, even more preferably between 2.5 and 5 mg/kg.

A pharmaceutical composition may additionally comprise one or more of the following substances, a detergent and/or a sugar. A preferred detergent is Tween 20. A preferred sugar is manitol.

The Fcγ receptor according to the invention preferably has sequence selected from the group of, SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12 and SEQ ID NO: 13. These are outlined in Table 1.

The Fcγ receptor according to the invention preferably comprises only the extra cellular domain of said sequences (soluble form of the receptor). Said domain is known from sequence alignments, is structurally characterized by x-ray crystallography and comprise the first two (CD16, CD32) or the first three (CD64) IgG-like domains of the mature receptor (Sondermann P., Kaiser J., Jacob U., Molecular basis for immune complex recognition: a comparison of Fc-receptor structures. J. Mol. Biol. 2001, 309, 737-749).

TABLE 1

| | | |
|---|---|---|
| Human | MWFLTTLLLWVPVDGQVDTTKAVIT | SEQ ID NO: 14 |
| FCgamma | LQPPWVSVFQEETVTLHCEVLHLPG | ATGTGGTTCTTGACAACTCTGCTCCTTTGGGTTCCAGT |
| RIA | SSSTQWFLNGTATQTSTPSY | TGATGGGCAAGTGGACACCACAAAGGCAGTGATCACTT |
| (SEQ ID | RITSASVNDSGEYRCQRGLSGRSDP | TGCAGCCTCCATGGGTCAGCGTGTTCCAAGAGGAAACC |
| NO: 1) | IQLEIHRGWLLLQVSSRVFTEGEPL | GTAACCTTGCACTGTGAGGTGCTCCATCTGCCTGGGAG |
| | ALRCHAWKDKLVYNVLYYRN | CAGCTCTACACAGTGGTTTCTCAATGGCACAGCCACTC |
| | GKAFKFFHWNSNLTILKTNISHNGT | AGACCTCGACCCCCAGCTACAGAATCACCTCTGCCAGT |
| | YHCSGMGKHRYTSAGISVTVKELFP | GTCAATGACAGTGGTGAATACAGGTGCCAGAGAGGTCT |
| | APVLNASVTSPLLEGNLVTL | CTCAGGGCGAAGTGACCCCATACAGCTGGAAATCCACA |
| | SCETKLLLQRPGLQLYFSFYMGSKT | GAGGCTGGCTACTACTGCAGGTCTCCAGCAGAGTCTTC |
| | LRGRNTSSEYQILTARREDSGLYWC | ACGGAAGGAGAACCTCTGGCCTTGAGGTGTCATGCGTG |
| | EAATEDGNVLKRSPELELQV | GAAGGATAAGCTGGTGTACAATGTGCTTTACTATCGAA |
| | LGLQLPTPVWFHVLFYLAVGIMFLV | ATGGCAAAGCCTTTAAGTTTTTCCACTGGAATTCTAAC |
| | NTVLWVTIRKELKRKKKWDLEISLD | CTCACCATTCTGAAAACCAACATAAGTCACAATGGCAC |
| | SGHEKKVISSLQEDRHLEEE | CTACCATTGCTCAGGCATGGGAAAGCATCGCTACACAT |
| | LKCQEQKEEQLQEGVHRKEPQGAT | CAGCAGGAATATCTGTCACTGTGAAAGAGCTATTTCCA |
| | | GCTCCAGTGCTGAATGCATCTGTGACATCCCCACTCCT |
| | | GGAGGGGAATCTGGTCACCCTGAGCTGTGAAACAAAGT |
| | | TGCTCTTGCAGAGGCCTGGTTTGCAGCTTTACTTCTCC |
| | | TTCTACATGGGCAGCAAGACCCTGCGAGGCAGGAACAC |
| | | ATCCTCTGAATACCAAATACTAACTGCTAGAAGAGAAG |
| | | ACTCTGGGTTATACTGGTGCGAGGCTGCCACAGAGGAT |
| | | GGAAATGTCCTTAAGCGCAGCCCTGAGTTGGAGCTTCA |
| | | AGTGCTTGGCCTCCAGTTACCAACTCCTGTCTGGTTTC |
| | | ATGTCCTTTTCTATCTGGCAGTGGGAATAATGTTTT |
| | | TAGTGAACACTGTTCTCTGGGTGACAATACGTAAAGAA |
| | | CTGAAAAGAAAGAAAAAGTGGGATTTAGAAATCTCTTT |
| | | GGATTCTGGTCATGAGAAGAAGGTAATTTCCAGCCTTC |
| | | AAGAAGACAGACATTTAGAAGAAGAGCTGAAATGTCAG |
| | | GAACAAAAAGAAGAACAGCTGCAGGAAGGGGTGCACCG |
| | | GAAGGAGCCCCAGGGGGCCACGTAG |
| Human | MWFLTTLLLWGWLLLQVSSRVFMEG | SEQ ID NO: 15 |
| FCgamma | EPLALRCHAWKDKLVYNVLYYRNGK | ATGTGGTTCTTGACAACTCTGCTCCTTTGGGGCTGGCT |
| RIB | AFKFFHWNSNLTILKTNISH | ACTACTGCAGGTCTCCAGCAGAGTCTTCATGGAAGGAG |
| (SEQ ID | NGTYHCSGMGKHRYTSAGISQYTVK | AACCTCTGGCCTTGAGGTGTCATGCGTGGAAGGATAAG |
| NO: 2) | GLQLPTPVWFHVLFYLAVGIMFLVN | CTGGTGTACAATGTGCTTTACTATCGAAATGGCAAAGC |
| | TVLWVTIRKELKRKKKWNLE | CTTTAAGTTTTTCCACTGGAATTCTAACCTCACCATTC |
| | ISLDSGHEKKVISSLQEDRHLEEEL | TGAAAACCAACATAAGTCACAATGGCACCTACCATTGC |
| | KCQEQKEEQLQEGVHRKEPQGAT | TCAGGCATGGGAAAGCATCGCTACACATCAGCAGGAAT |
| | | ATCACAATACACTGTGAAAGGCCTCCAGTTACCAACTC |
| | | CTGTCTGGTTTCATGTCCTTTTCTATCTGGCAGTGGGA |
| | | ATAATGTTTTTAGTGAACACTGTTCTCTGGGTGACAAT |
| | | ACGTAAAGAACTGAAAAGAAAGAAAAAGTGGAATTTAG |
| | | AAATCTCTTTGGATTCTGGTCATGAGAAGAAGGTAATT |
| | | TCCAGCCTTCAAGAAGACAGACATTTAGAAGAAGAGCT |
| | | GAAATGTCAGGAACAAAAAGAAGAACAGCTGCAGGAAG |
| | | GGGTGCACCGGAAGGAGCCCCAGGGGGCCACGTAG |
| Human | MWFLTTLLLWVPVDGQVDTTKAVIT | SEQ ID NO: 16 |
| FCgamma | LQPPWVSVFQEETVTLHCEVLHLPG | ATGTGGTTCTTGACAACTCTGCTCCTTTGGGTTCCAGT |
| RIB | SSSTQWFLNGTATQTSTPSY | TGATGGGCAAGTGGACACCACAAAGGCAGTGATCACTT |
| (SEQ ID | RITSASVNDSGEYRCQRGLSGRSDP | TGCAGCCTCCATGGGTCAGCGTGTTCCAAGAGGAAACC |
| NO: 3) | IQLEIHRGWLLLQVSSRVFMEGEPL | GTAACCTTGCACTGTGAGGTGCTCCATCTGCCTGGGAG |
| | ALRCHAWKDKLVYNVLYYRN | CAGCTCCACACAGTGGTTTCTCAATGGCACAGCCACTC |
| | GKAFKFFHWNSNLTILKTNISHNGT | AGACCTCGACCCCCAGCTACAGAATCACCTCTGCCAGT |
| | YHCSGMGKHRYTSAGISQYTVKGLQ | GTCAATGACAGTGGTGAATACAGGTGCCAGAGAGGTCT |
| | LPTPVWFHVLFYLAVGIMFL | CTCAGGGCGAAGTGACCCCATACAGCTGGAAATCCACA |
| | VNTVLWVTIRKELKRKKKWNLEISL | GAGGCTGGCTACTACTGCAGGTCTCCAGCAGAGTCTTC |
| | DSGHEKKVISSLQEDRHLEEELKCQ | ATGGAAGGAGAACCTCTGGCCTTGAGGTGTCATGCGTG |
| | EQKEEQLQEGVHRKEPQGAT | GAAGGATAAGCTGGTGTACAATGTGCTTTACTATCGAA |
| | | ATGGCAAAGCCTTTAAGTTTTTCCACTGGAATTCTAAC |
| | | CTCACCATTCTGAAAACCAACATAAGTCACAATGGCAC |
| | | CTACCATTGCTCAGGCATGGGAAAGCATCGCTACACAT |
| | | CAGCAGGAATATCACAATACACTGTGAAAGGCCTCCAG |
| | | TTACCAACTCCTGTCTGGTTTCATGTCCTTTTCTATCT |
| | | GGCAGTGGGAATAATGTTTTTAGTGAACACTGTTCTCT |
| | | GGGTGACAATACGTAAAGAACTGAAAAGAAAGAAAAAG |
| | | TGGAATTTAGAAATCTCTTTGGATTCTGGTCATGAGAA |
| | | GAAGGTAATTTCCAGCCTTCAAGAAGACAGACATTTAG |
| | | AAGAAGAGCTGAAATGTCAGGAACAAAAAGAAGAACAG |
| | | CTGCAGGAAGGGGTGCACCGGAAGGAGCCCCAGGGGGC |
| | | CACGTAG |
| Human | MTMETQMSQNVCPRNLWLLQPLTVL | SEQ ID NO: 17 |
| FCgamma | LLLASADSQAAPPKAVLKLEPPWIN | ATGACTATGGAGACCCAAATGTCTCAGAATGTATGTCC |
| RIIa | VLQEDSVTLTCQGARSPESD | CAGAAACCTGTGGCTGCTTCAACCATTGACAGTTTTGC |
| (SEQ ID | SIQWFHNGNLIPTHTQPSYRFKANN | TGCTGCTGGCTTCTGCAGACAGTCAAGCTGCTCCCCCA |
| NO: 4) | NDSGEYTCQTGQTSLSDPVHLTVLS | AAGGCTGTGCTGAAACTTGAGCCCCCGTGGATCAACGT |
| | EWLVLQTPHLEFQEGETIML | GCTCCAGGAGGACTCTGTGACTCTGACATGCCAGGGGG |
| | RCHSWKDKPLVKVTFFQNGKSQKFS | CTCGCAGCCCTGAGAGCGACTCCATTCAGTGGTTCCAC |
| | HLDPTFSIPQANHSHSGDYHCTGNI | AATGGGAATCTCATTCCCACCCACACGCAGCCCAGCTA |
| | GYTLFSSKPVTITVQVPSMG | CAGGTTCAAGGCCAACAACAATGACAGCGGGGAGTACA |
| | SSSPMGIIVAVVIATAVAAIVAAVV | CGTGCCAGACTGGCCAGACCAGCCTCAGCGACCCTGTG |
| | ALIYCRKKRISANSTDPVKAAQFEP | CATCTGACTGTGCTTTCCGAATGGCTGGTGCTCCAGAC |
| | PGRQMIAIRKRQLEETNNDY | CCCTCACCTGGAGTTCCAGGAGGGAGAAACCATCATGC |
| | ETADGGYMTLNPRAPTDDDKNIYLT | TGAGGTGCCACAGCTGGAAGGACAAGCCTCTGGTCAAG |
| | LPPNDHVNSNN | GTCACATTCTTCCAGAATGGAAAATCCCAGAAATTCTC |
| | | CCATTTGGATCCCACCTTCTCCATCCCACAAGCAAACC |
| | | ACAGTCACAGTGGTGATTACCACTGCACAGGAAACATA |
| | | GGCTACACGCTGTTCTCATCCAAGCCTGTGACCATCAC |
| | | TGTCCAAGTGCCCAGCATGGGCAGCTCTTCACCAATGG |
| | | GGATCATTGTGGCTGTGGTCATTGCGACTGCTGTAGCA |
| | | GCCATTGTTGCTGCTGTAGTGGCCTTGATCTACTGCAG |
| | | GAAAAAGCGGATTTCAGCCAATTCCACTGATCCTGTGA |
| | | AGGCTGCCCAATTTGAGCCACCTGGACGTCAAATGATT |
| | | GCCATCAGAAAGAGACAACTTGAAGAAACCAACAATGA |
| | | CTATGAAACAGCTGACGGCGGCTACATGACTCTGAACC |
| | | CCAGGGCACCTACTGACGATGATAAAAACATCTACCTG |
| | | ACTCTTCCTCCCAACGACCATGTCAACAGTAATAACTA |
| | | A |
| Human | MGILSFLPVLATESDWADCKSPQPW | SEQ ID NO: 18 |
| FCgamma | GHMLLWTAVLFLAPVAGTPAAPPKA | ATGGGAATCCTGTCATTCTTACCTGTCCTTGCCACTGA |
| RIIB | VLKLEPQWINVLQEDSVTLT | GAGTGACTGGGCTGACTGCAAGTCCCCCCAGCCTTGGG |
| Isoform | CRGTHSPESDSIQWFHNGNLIPTHT | GTCATATGCTTCTGTGGACAGCTGTGCTATTCCTGGCT |
| 1 | QPSYRFKANNNDSGEYTCQTGQTSL | CCTGTTGCTGGGACACCTGCAGCTCCCCCAAAGGCTGT |
| (SEQ ID | SDPVHLTVLSEWLVLQTPHL | GCTGAAACTCGAGCCCCAGTGGATCAACGTGCTCCAGG |
| NO: 5) | EFQEGETIVLRCHSWKDKPLVKVTF | AGGACTCTGTGACTCTGACATGCCGGGGGACTCACAGC |
| | FQNGKSKKFSRSDPNFSIPQANHSH | CCTGAGAGCGACTCCATTCAGTGGTTCCACAATGGGAA |
| | SGDYHCTGNIGYTLYSSKPV | TCTCATTCCCACCCACACGCAGCCCAGCTACAGGTTCA |
| | TITVQAPSSSPMGIIVAVVTGIAVA | AGGCCAACAACAATGACAGCGGGGAGTACACGTGCCAG |
| | AIVAAVVALIYCRKKRISALPGYPE | ACTGGCCAGACCAGCCTCAGCGACCCTGTGCATCTGAC |
| | CREMGETLPEKPANPTNPDE | TGTGCTTTCTGAGTGGCTGGTGCTCCAGACCCCTCACC |
| | ADKVGAENTITYSLLMHPDALEEPD | TGGAGTTCCAGGAGGGAGAAACCATCGTGCTGAGGTGC |
| | DQNRI | CACAGCTGGAAGGACAAGCCTCTGGTCAAGGTCACATT |
| | | CTTCCAGAATGGAAAATCCAAGAAATTTTCCCGTTCGG |
| | | ATCCCAACTTCTCCATCCCACAAGCAAACCACAGTCAC |
| | | AGTGGTGATTACCACTGCACAGGAAACATAGGCTACAC |
| | | GCTGTACTCATCCAAGCCTGTGACCATCACTGTCCAAG |
| | | CTCCCAGCTCTTCACCGATGGGGATCATTGTGGCTGTG |
| | | GTCACTGGGATTGCTGTAGCGGCCATTGTTGCTGCTGT |
| | | AGTGGCCTTGATCTACTGCAGGAAAAAGCGGATTTCAG |
| | | CTCTCCCAGGATACCCTGAGTGCAGGGAAATGGGAGAG |
| | | ACCCTCCCTGAGAAACCAGCCAATCCCACTAATCCTGA |
| | | TGAGGCTGACAAAGTTGGGGCTGAGAACACAATCACCT |
| | | ATTCACTTCTCATGCACCCGGATGCTCTGGAAGAGCCT |
| | | GATGACCAGAACCGTATTTAG |
| Human | MGILSFLPVLATESDWADCKSPQPW | SEQ ID NO: 19 |
| FCgamma | GHMLLWTAVLFLAPVAGTPAPPKAV | tctagcgagg tgacagcgta gaaccagaga |
| RIIB | LKLEPQWINVLQEDSVTLTC | atttgtttgc cctctagggt agaatccgcc |
| Isoform | RGTHSPESDSIQWFHNGNLIPTHTQ | aagctttgag agaaggctgt gactgctgtg |
| 2 | PSYRFKANNNDSGEYTCQTGQTSLS | ctctgggcgc cagctcgctc cagggagtgg |
| (SEQ ID | DPVHLTVLSEWLVLQTPHLE | tgggaatcct gtcattctta cctgtccttg |
| NO: 6) | FQEGETIVLRCHSWKDKPLVKVTFF | ccactgagag tgactgggct gactgcaagt |
| | QNGKSKKFSRSDPNFSIPQANHSHS | ccccccagcc ttggggtcat atgcttctgt |
| | GDYHCTGNIGYTLYSSKPVT | ggacagctgt gctattcctg gctcctgttg |
| | ITVQAPSSSPMGIIVAVVTGIAVAA | ctgggacacc tgcagctccc ccaaaggctg |
| | IVAAVVALIYCRKKRISANPTNPDE | tgctgaaact cgagccccag tggatcaacg |
| | ADKVGAENTITYSLLMHPDA | tgctccagga ggactctgtg actctgacat |
| | LEEPDDQNRI | gccgggggac tcacagccct gagagcgact |
| | | ccattcagtg gttccacaat gggaatctca |
| | | ttcccaccca cacgcagccc agctacaggt |
| | | tcaaggccaa caacaatgac agcggggagt |
| | | acacgtgcca gactggccag accagcctca |
| | | gcgaccctgt gcatctgact gtgctttctg |
| | | agtggctggt gctccagacc cctcacctgg |
| | | agttccagga gggagaaacc atcgtgctga |
| | | ggtgccacag ctggaaagac aagcctctgg |
| | | tcaaggtcac attcttccag aatggaaaat |
| | | ccaagaaatt ttcccgttcg gatcccaact |
| | | tctccatccc acaagcaaac cacagtccca |
| | | gtgggtgatt accactgcac aggaaaaaat |
| | | agggctacac cctgtactca tccaagccct |
| | | gtggaccatc actgttcaaa gctccccaac |
| | | ttcttcacgg atggggga |
| Human | MGILSFLPVLATESDWADCKSPQPW | SEQ ID NO: 20 |
| FCgamma | GHMLLWTAVLFLAPVAGTPAAPPKA | ATGGGAATCCTGTCATTCTTACCTGTCCTTGCCACTGA |
| RIIB | VLKLEPQWINVLQEDSVTLT | GAGTGACTGGGCTGACTGCAAGTCCCCCCAGCCTTGGG |
| Isoform | CRGTHSPESDSIQWFHNGNLIPTHT | GTCATATGCTTCTGTGGACAGCTGTGCTATTCCTGGCT |
| 3 | QPSYRFKANNNDSGEYTCQTGQTSL | CCTGTTGCTGGGACACCTGCAGCTCCCCCAAAGGCTGT |
| (SEQ ID | SDPVHLTVLSEWLVLQTPHL | GCTGAAACTCGAGCCCCAGTGGATCAACGTGCTCCAGG |
| NO: 7) | EFQEGETIVLRCHSWKDKPLVKVTF | AGGACTCTGTGACTCTGACATGCCGGGGGACTCACAGC |
| | FQNGKSKKFSRSDPNFSIPQANHSH | CCTGAGAGCGACTCCATTCAGTGGTTCCACAATGGGAA |
| | SGDYHCTGNIGYTLYSSKPV | TCTCATTCCCACCCACACGCAGCCCAGCTACAGGTTCA |
| | TITVQAPSSSPMGIIVAVVTGIAVA | AGGCCAACAACAATGACAGCGGGGAGTACACGTGCCAG |
| | AIVAAVVALIYCRKKRISANPTNPD | ACTGGCCAGACCAGCCTCAGCGACCCTGTGCATCTGAC |
| | EADKVGAENTITYSLLMHPD | TGTGCTTTCTGAGTGGCTGGTGCTCCAGACCCCTCACC |
| | ALEEPDDQNRI | TGGAGTTCCAGGAGGGAGAAACCATCGTGCTGAGGTGC |
| | | CACAGCTGGAAGGACAAGCCTCTGGTCAAGGTCACATT |
| | | CTTCCAGAATGGAAAATCCAAGAAATTTTCCCGTTCGG |
| | | ATCCCAACTTCTCCATCCCACAAGCAAACCACAGTCAC |
| | | AGTGGTGATTACCACTGCACAGGAAACATAGGCTACAC |
| | | GCTGTACTCATCCAAGCCTGTGACCATCACTGTCCAAG |
| | | CTCCCAGCTCTTCACCGATGGGGATCATTGTGGCTGTG |
| | | GTCACTGGGATTGCTGTAGCGGCCATTGTTGCTGCTGT |
| | | AGTGGCCTTGATCTACTGCAGGAAAAAGCGGATTTCAG |
| | | CCAATCCCACTAATCCTGATGAGGCTGACAAAGTTGGG |
| | | GCTGAGAACACAATCACCTATTCACTTCTCATGCACCC |
| | | GGATGCTCTGGAAGAGCCTGATGACCAGAACCGTATTT |
| | | AG |
| Human | mgilsflpvl atesdwadck | SEQ ID NO: 21 |
| FCgamma | spqpwghmll wtavlflapv | atgggaatcc tgtcattctt acctgtcctt |
| RIIb | agtpappkav lklepqwinv | gccactgaga gtgactgggc tgactgcaag |
| Isoform | lqedsvtltc rgthspesds | tccccccagc cttggggtca tatgcttctg |
| 4 | iqwfhngnli pthtqpsyrf | tggacagctg tgctattcct ggctcctgtt |
| (SEQ ID | kannndsgey tcqtgqtsls | gctgggacac ctgctccccc aaaggctgtg |
| NO: 8) | dpvhltvlse wlvlqtphle | ctgaaactcg agccccagtg gatcaacgtg |
| | fqegetivlr chswkdkplv | ctccaggagg actctgtgac tctgacatgc |
| | kvtffqngks kkfsrsdpnf | cgggggactc acagccctga gagcgactcc |
| | sipqanhshs gdyhctgnig | attcagtggt tccacaatgg gaatctcatt |
| | ytlysskpvt itvqapsssp | cccacccaca cgcagcccag ctacaggttc |
| | mgiivavvtg iavaaivaav | aaggccaaca acaatgacag cggggagtac |
| | valiycrkkr isalpgypec | acgtgccaga ctggccagac cagcctcagc |
| | remgetlpek panptnpdea | gaccctgtgc atctgactgt gctttctgag |
| | dkvgaentit ysllmhpdal | tggctggtgc tccagacccc tcacctggag |
| | eepddqnri | ttccaggagg gagaaaccat cgtgctgagg |
| | | tgccacagct ggaaggacaa gcctctggtc |
| | | aaggtcacat tcttccagaa tggaaaatcc |
| | | aagaaatttt cccgttcgga tcccaacttc |
| | | tccatcccac aagcaaacca cagtcacagt |
| | | ggtgattacc actgcacagg aaacataggc |
| | | tacacgctgt actcatccaa gcctgtgacc |
| | | atcactgtcc aagctcccag ctcttcaccg |
| | | atggggatca ttgtggctgt ggtcactggg |
| | | attgctgtag cggccattgt tgctgctgta |
| | | gtggccttga tctactgcag gaaaaagcgg |
| | | atttcagctc tcccaggata ccctgagtgc |
| | | agggaaatgg gagagaccct ccctgagaaa |
| | | ccagccaatc ccactaatcc tgatgaggct |
| | | gacaaagttg gggctgagaa cacaatcacc |
| | | tattcacttc tcatgcaccc ggatgctctg |
| | | gaagagcctg atgaccagaa ccgtatttag |
| Human | MGILSFLPVLATESDWADCKSPQPW | SEQ ID NO: 22 |
| FCgamma | GHMLLWTAVLFLAPVAGTP | atgggaatcc tgtcattctt acctgtcctt |
| RIIc | AAPPKAVLKLEPQWINVLQEDSVTL | gccactgaga gtgactgggc tgactgcaag |
| Isoform | TCRGTHSPESDSIPWFHNGNLIPTH | tccccccagc cttggggtca tatgcttctg |
| 1 | TQPSYRFK | tggacagctg tgctattcct ggctcctgtt |
| (SEQ ID | ANNNDSGEYTCQTGQTSLSDPVHLT | gctgggacac ctgcagctcc cccaaaggct |
| NO: 9) | VLSEWLVLQTPHLEFQEGETIVLRC | gtgctgaaac tcgagcccca gtggatcaac |
| | HSWKDKPL | gtgctccaag aggactctgt gactctgaca |
| | VKVTFFQNGKSKKFSRSDPNFSIPQ | tgccggggga ctcacagccc tgagagcgac |
| | ANHSHSGDYHCTGNIGYTLYSSKPV | tccattccgt ggttccacaa tgggaatctc |
| | TITVQAPS | attcccaccc acacgcagcc cagctacagg |
| | SSPMGIIVAVVTGIAVAAIVAAVVA | ttcaaggcca acaacaatga cagcggggag |
| | LIYCRKKRISANSTDPVKAAQFEPP | tacacgtgcc agactggcca gaccagcctc |
| | GRQMIAIR | agcgaccctg tgcatctgac tgtgctttct |
| | KRQPEETNNDYETADGGYMTLNPRA | gagtggctgg tgctccagac ccctcacctg |
| | PTDDDKNIYLTLPPNDHVNSNN | gagttccagg agggagaaac catcgtgctg |
| | | aggtgccaca gctggaagga caagcctctg |
| | | gtcaaggtca cattcttcca gaatggaaaa |
| | | tccaagaaat tttcccgttc ggatcccaac |
| | | ttctccatcc cacaagcaaa ccacagtcac |
| | | agtggtgatt accactgcac aggaaacata |
| | | ggctacacgc tgtactcatc caagcctgtg |
| | | accatcactg tccaagctcc cagctcttca |
| | | ccgatgggga tcattgtggc tgtggtcact |
| | | gggattgctg tagcggccat tgttgctgct |
| | | gtagtggcct tgatctactg caggaaaaag |
| | | cggatttcag ccaattccac tgatcctgtg |
| | | aaggctgccc aatttgagcc acctggacgt |
| | | caaatgattg ccatcagaaa gagacaacct |
| | | gaagaaacca acaatgacta tgaaacagct |
| | | gacggcggct acatgactct gaaccccagg |
| | | gcacctactg acgatgataa aaacatctac |
| | | ctgactcttc ctcccaacga ccatgtcaac |
| | | agtaataact aa |
| Human | MGILSFLPVLATESDWADCKSPQPW | SEQ ID NO: 23 |
| FCgamma | GHMLLWTAVLFLAPVAGTP | atgggaatcc tgtcattctt acctgtcctt |
| RIIC | AAPPKAVLKLEPQWINVLQEDSVTL | gccactgaga gtgactgggc tgactgcaag |
| Isoform | TCRGTHSPESDSIPWFHNGNLIPTH | tccccccagc cttggggtca tatgcttctg |
| 2 | TQPSYRFK | tggacagctg tgctattcct ggctcctgtt |
| (SEQ ID | ANNNDSGEYTCQTGQTSLSDPVHLT | gctgggacac ctgcagctcc cccaaaggct |
| NO: 10) | VLSEWLVLQTPHLEFQEGETIVLRC | gtgctgaaac tcgagcccca gtggatcaac |
| | HSWKDKPL | gtgctccaag aggactctgt gactctgaca |
| | VKVTFFQNGKSKKFSRSDPNFSIPQ | tgccggggga ctcacagccc tgagagcgac |
| | ANHSHSGDYHCTGNIGYTLYSSKPV | tccattccgt ggttccacaa tgggaatctc |
| | TITVQAPS | attcccaccc acacgcagcc cagctacagg |
| | SSPMGIIVAVVTGIAVAAIVAAVVA | ttcaaggcca acaacaatga cagcggggag |
| | LIYCRKKRISANSTDPVKAAQFEML | tacacgtgcc agactggcca gaccagcctc |
| | SCTHLDVK | agcgaccctg tgcatctgac tgtgctttct |
| | | gagtggctgg tgctccagac ccctcacctg |
| | | gagttccagg agggagaaac catcgtgctg |
| | | aggtgccaca gctggaagga caagcctctg |
| | | gtcaaggtca cattcttcca gaatggaaaa |
| | | tccaagaaat tttcccgttc ggatcccaac |
| | | ttctccatcc cacaagcaaa ccacagtcac |
| | | agtggtgatt accactgcac aggaaacata |
| | | ggctacacgc tgtactcatc caagcctgtg |
| | | accatcactg tccaagctcc cagctcttca |
| | | ccgatgggga tcattgtggc tgtggtcact |
| | | gggattgctg tagcggccat tgttgctgct |
| | | gtagtggcct tgatctactg caggaaaaag |
| | | cggatttcag ccaattccac tgatcctgtg |
| | | aaggctgccc aatttgagat gctttcctgc |
| | | acccacctgg acgtcaaatg a |
| Human | MGILSFLPVLATESDWADCKSPQPW | SEQ ID NO: 24 |
| FCgamma | GHMLLWTAVLFLAPVAGTP | atgggaatcc tgtcattctt acctgtcctt |
| RIIC | AAPPKAVLKLEPQWINVLQEDSVTL | gccactgaga gtgactgggc tgactgcaag |
| Isoform | TCRGTHSPESDSIPWFHNGNLIPTH | tccccccagc cttggggtca tatgcttctg |
| 3 | TQPSYRFK | tggacagctg tgctattcct ggctcctgtt |
| (SEQ ID | ANNNDSGEYTCQTGQTSLSDPVHLT | gctgggacac ctgcagctcc cccaaaggct |
| NO: 11) | VLSEWLVLQTPHLEFQEGETIVLRC | gtgctgaaac tcgagcccca gtggatcaac |
| | HSWKDKPL | gtgctccaag aggactctgt gactctgaca |
| | VKVTFFQNGKSKKFSRSDPNFSIPQ | tgccggggga ctcacagccc tgagagcgac |
| | ANHSHSGDYHCTGNIGYTLYSSKPV | tccattccgt ggttccacaa tgggaatctc |
| | TITVQAPS | attcccaccc acacgcagcc cagctacagg |
| | SSPMGIIVAVVTGIAVAAIVAAVVA | ttcaaggcca acaacaatga cagcggggag |
| | LIYCRKKRISATWTSNDCHQKETT | tacacgtgcc agactggcca gaccagcctc |
| | | agcgaccctg tgcatctgac tgtgctttct |
| | | gagtggctgg tgctccagac ccctcacctg |
| | | gagttccagg agggagaaac catcgtgctg |
| | | aggtgccaca gctggaagga caagcctctg |
| | | gtcaaggtca cattcttcca gaatggaaaa |
| | | tccaagaaat tttcccgttc ggatcccaac |
| | | ttctccatcc cacaagcaaa ccacagtcac |
| | | agtggtgatt accactgcac aggaaacata |
| | | ggctacacgc tgtactcatc caagcctgtg |
| | | accatcactg tccaagctcc cagctcttca |
| | | ccgatgggga tcattgtggc tgtggtcact |
| | | gggattgctg tagcggccat tgttgctgct |
| | | gtagtggcct tgatctactg caggaaaaag |
| | | cggatttcag ccacctggac gtcaaatgat |
| | | tgccatcaga aagagacaac ctga |
| Human | MGGGAGERLFTSSCLVGLVPLGLRI | SEQ ID NO: 25 |
| FCgamma | SLVTCPLQCGIMWQLLLPTALLLLV | ATGGGTGGAGGGGCTGGGGAAAGGCTGTTTACTTCCTC |
| RIIIA | SAGMRTEDLPKAVVFLEPQW | CTGTCTAGTCGGTTTGGTCCCTTTAGGGCTCCGGATAT |
| (SEQ ID | YRVLEKDSVTLKCQGAYSPEDNSTQ | CTTTGGTGACTTGTCCACTCCAGTGTGGCATCATGTGG |
| NO: 12) | WFHNESLISSQASSYFIDAATVDDS | CAGCTGCTCCTCCCAACTGCTCTGCTACTTCTAGTTTC |
| | GEYRCQTNLSTLSDPVQLEV | AGCTGGCATGCGGACTGAAGATCTCCCAAAGGCTGTGG |
| | HIGWLLLQAPRWVFKEEDPIHLRCH | TGTTCCTGGAGCCTCAATGGTACAGGGTGCTCGAGAAG |
| | SWKNTALHKVTYLQNGKGRKYFHHN | GACAGTGTGACTCTGAAGTGCCAGGGAGCCTACTCCCC |
| | SDFYIPKATLKDSGSYFCRG | TGAGGACAATTCCACACAGTGGTTTCACAATGAGAGCC |
| | LFGSKNVSSETVNITITQGLAVSTI | TCATCTCAAGCCAGGCCTCGAGCTACTTCATTGACGCT |
| | SSFFPPGYQVSFCLVMVLLFAVDTG | GCCACAGTCGACGACAGTGGAGAGTACAGGTGCCAGAC |
| | LYFSVKTNIRSSTRDWKDHK | AAACCTCTCCACCCTCAGTGACCCGGTGCAGCTAGAAG |
| | FKWRKDPQDK | TCCATATCGGCTGGCTGTTGCTCCAGGCCCCTCGGTGG |
| | | GTGTTCAAGGAGGAAGACCCTATTCACCTGAGGTGTCA |
| | | CAGCTGGAAGAACACTGCTCTGCATAAGGTCACATATT |
| | | TACAGAATGGCAAAGGCAGGAAGTATTTTCATCATAAT |
| | | TCTGACTTCTACATTCCAAAAGCCACACTCAAAGACAG |
| | | CGGCTCCTACTTCTGCAGGGGGCTTTTTGGGAGTAAAA |
| | | ATGTGTCTTCAGAGACTGTGAACATCACCATCACTCAA |
| | | GGTTTGGCAGTGTCAACCATCTCATCATTCTTTCCACC |
| | | TGGGTACCAAGTCTCTTTCTGCTTGGTGATGGTACTCC |
| | | TTTTTGCAGTGGACACAGGACTATATTTCTCTGTGAAG |
| | | ACAAACATTCGAAGCTCAACAAGAGACTGGAAGGACCA |
| | | TAAATTTAAATGGAGAAAGGACCCTCAAGACAAATGA |
| Human | MWQLLLPTALLLLVSAGMRTEDLPK | SEQ ID NO: 26 |
| FCgamma | AVVFLEPQWYSVLEKDSVTLKCQGA | ATGTGGCAGCTGCTCCTCCCAACTGCTCTGCTACTTCT |
| RIIIB | YSPEDNSTQWFHNENLISSQ | AGTTTCAGCTGGCATGCGGACTGAAGATCTCCCAAAGG |
| (SEQ ID | ASSYFIDAATVNDSGEYRCQTNLST | CTGTGGTGTTCCTGGAGCCTCAATGGTACAGCGTGCTT |
| NO: 13) | LSDPVQLEVHIGWLLLQAPRWVFKE | GAGAAGGACAGTGTGACTCTGAAGTGCCAGGGAGCCTA |
| | EDPIHLRCHSWKNTALHKVT | CTCCCCTGAGGACAATTCCACACAGTGGTTTCACAATG |
| | YLQNGKDRKYFHHNSDFHIPKATLK | AGAACCTCATCTCAAGCCAGGCCTCGAGCTACTTCATT |
| | DSGSYFCRGLVGSKNVSSETVNITI | GACGCTGCCACAGTCAACGACAGTGGAGAGTACAGGTG |
| | TQGLAVSTISSFSPPGYQVS | CCAGACAAACCTCTCCACCCTCAGTGACCCGGTGCAGC |
| | FCLVMVLLFAVDTGLYFSVKTNI | TAGAAGTCCATATCGGCTGGCTGTTGCTCCAGGCCCCT |
| | | CGGTGGGTGTTCAAGGAGGAAGACCCTATTCACCTGAG |
| | | GTGTCACAGCTGGAAGAACACTGCTCTGCATAAGGTCA |
| | | CATATTTACAGAATGGCAAAGACAGGAAGTATTTTCAT |
| | | CATAATTCTGACTTCCACATTCCAAAAGCCACACTCAA |
| | | AGATAGCGGCTCCTACTTCTGCAGGGGGCTTGTTGGGA |
| | | GTAAAAATGTGTCTTCAGAGACTGTGAACATCACCATC |
| | | ACTCAAGGTTTGGCAGTGTCAACCATCTCATCATTCTC |
| | | TCCACCTGGGTACCAAGTCTCTTTCTGCTTGGTGATGG |
| | | TACTCCTTTTTGCAGTGGACACAGGACTATATTTCTCT |
| | | GTGAAGACAAACATTTGA |

The preferred FcRs are encoded by the following nucleic acids:

Human FCgammaRIA (SEQ ID NO: 1) is encoded by the sequence according to SEQ ID NO: 14.

Human FCgammaRIB (SEQ ID NO: 2) is encoded by the sequence according to SEQ ID NO: 15.

Human FCgammaRIB (SEQ ID NO: 3) is encoded by the sequence according to SEQ ID NO: 16.

Human FCgammaRIIa (SEQ ID NO: 4) is encoded by the sequence according to SEQ ID NO: 17.

Human FCgammARIIB Isoform 1 (SEQ ID NO: 5) is encoded by the sequence according to SEQ ID NO: 18.

Human FCgammaRIIB Isoform 2 (SEQ ID NO: 6) is encoded by the sequence according to SEQ ID NO: 19.

Human FCgammaRIIB Isoform 3 (SEQ ID NO: 7) is encoded by the sequence according to SEQ ID NO: 20.

Human FCgammaRIIb Isoform 4 (SEQ ID NO: 8) is encoded by the sequence according to SEQ ID NO: 21.

Human FCgammaRIIc Isoform 1 (SEQ ID NO: 9) is encoded by the sequence according to SEQ ID NO: 22.

Human FCgammaRIIC Isoform 2 (SEQ ID NO: 10) is encoded by the sequence according to SEQ ID NO: 23.

Human FCgammaRIIC Isoform 3 (SEQ ID NO: 11) is encoded by the sequence according to SEQ ID NO: 24.

Human FCgammaRIIIA (SEQ ID NO: 12) is encoded by the sequence according to SEQ ID NO: 25.

Human FCgammaRIIIB (SEQ ID NO: 13) is encoded by the sequence according to SEQ ID NO: 26.

It is preferred that the FcγR is a recombinant non-glycosylated human soluble FcγRIIb preferably selected from the group of SEQ ID NO: 5, SEQ ID NO: 6, SEQ ID NO: 7 and SEQ ID NO: 8.

### EXAMPLES

The biologically active compound, the recombinant human soluble FcγRIIb molecule, is produced in inclusion bodies by fermentation of genetically engineered E. coli.

sFcγRIIb is produced by fermentation in E. coli strain BL21 (DE3) which has been transformed with an optimized cDNA sequence for the expression of sFcγRIIb.

The sFcγRIIb contains 4 cysteins to form 2 intra-molecular disulfide bonds between positions Cys26-Cys68 and Cys107-Cys151. The N-terminal sequence corresponds to the consensus sequence of the eukaryotic signal peptidase. The C-terminus was generated by introduction of a stop codon after the SER-SER-PRO motive. No further modifications were introduced.

The sFcγRIIb has a molecular mass of 19,688.9 at native and of 19,692.9 at reducing conditions.

An overview of the manufacturing process of the drug substance is given in Table 2:

TABLE 2

One vial of WCB
Fermentation of E. coli cell substrate
Harvest of E. coli cell substrate
Isolation of inclusion bodies
Refolding of sFcγRIIb from inclusion bodies
Purification of sFcγRIIb to yield bulk material
Storage of sFcγRIIb bulk at −80° C.

### Figure Captions

FIG. 1

EAE was induced on day zero in 6-8 week old C57/B16j female mice by subcutaneous injection of 100 μg rat MOG in complete Freund adjuvant (CFA). 250 ng of pertussistoxin were given intraperitoneally on day 0 and day 2. The mice were treated intraperitoneally with 200 μg sFcR after first symptoms appeared on day 8, 11 and 14. The scoring scheme was according to the degree of paralysis: 0=no paralysis, 1=tail paralysis, 2=hind legs paralysis, 3=front legs paralysis, 4=complete paralysis, 5=death.

FIG. 2

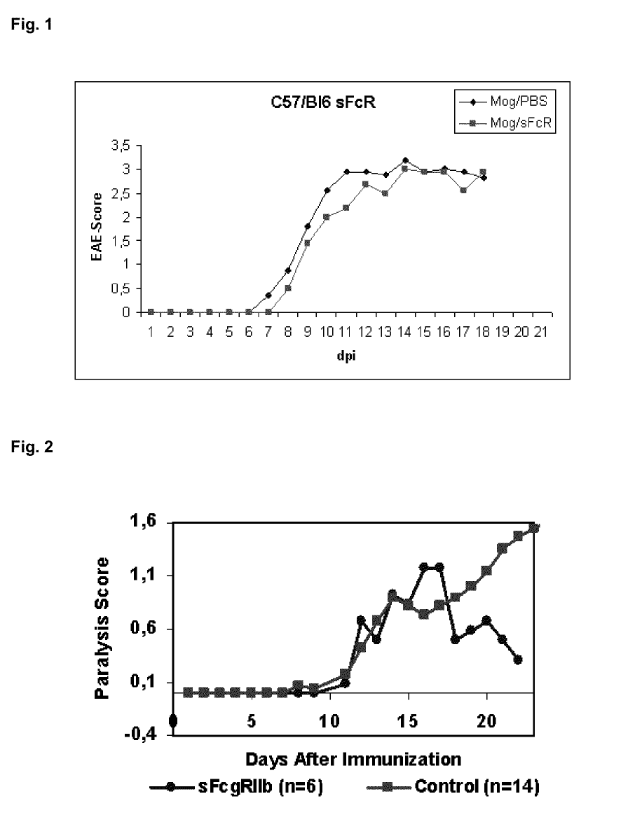

EAE was induced on day zero in 6-8 week old SJL female mice by subcutaneous injection of 100 μg rat MOG in complete Freund adjuvant (CFA). 250 ng of pertussistoxin were given intraperitoneally on day 0 and day 2. The mice were treated intraperitoneally with 200 μg sFcR after first symptoms appeared on day 9, 12 and 15. The scoring scheme was according to the degree of paralysis: 0=no paralysis, 1=tail paralysis, 2=hind legs paralysis, 3=front legs paralysis, 4=complete paralysis, 5=death.

1-11. (canceled)

12. A method for treating multiple sclerosis in a patient comprising administering to said patient a soluble Fcγ receptor (Fc-gamma receptor) for use in treating multiple sclerosis, wherein the multiple sclerosis is a B cell mediated form of multiple sclerosis and/or an autoantibody driven form of multiple sclerosis.

13. The method according to claim 12, wherein the B cell mediation of the multiple sclerosis and/or autoantibody driven form of multiple sclerosis is characterized by one or more of the following features:
(a) the multiple sclerosis is ameliorated if the patient undergoes intravenous immunoglobulin (IVIG) treatment;
(b) the multiple sclerosis is ameliorated if the patient undergoes anti-CD20 antibody treatment;
(c) the multiple sclerosis is ameliorated if the patient undergoes plasmapheresis;
(d) the multiple Sclerosis is ameliorated if the patient undergoes immunoadsorption;
(e) the presence of autoantibodies against the antigen myelin oligodendrocyte glycoprotein (MOG);
(f) the presence of autoantibodies against the antigen myelin basic protein (MBP); or
(g) the presence of autoantibodies against aquaporin 4.

14. The method according to claim 12, wherein the B cell mediation of the multiple sclerosis and/or autoantibody driven form of multiple sclerosis is determined prior to the use of the Fcγ receptor by means of one or more of the following tests:
(a) determining whether the multiple sclerosis is ameliorated if the patient undergoes intravenous immunoglobulin (IVIG) treatment;
(b) determining whether the multiple sclerosis is ameliorated if the patient undergoes anti-CD20 antibody treatment;
(c) determining whether the multiple sclerosis is ameliorated if the patient undergoes plasmapheresis;
(d) the multiple sclerosis is ameliorated if the patient undergoes immunoadsorption;
(e) determining whether autoantibodies against the antigen myelin mligodendrocyte glycoprotein (MOG) are present in the patient;
(f) determining whether autoantibodies against the antigen myelin basic protein (MBP) are present in the patient; or
(g) determining whether autoantibodies against aquaporin 4 are present in the patient.

15. The method according to claim 12, wherein the Fcγ is selected from the group of, FcγRI (CD64), FcγRIIA (CD32), FcγRIIB1 (CD32), FcγRIIB2 (CD32), FcγRIIc (CD32), FcγRIIIA (CD16) and FcγRIIIB (CD16).

16. The method according to claim 12, wherein the receptor is chemically modified by PEGylation and/or affinity modulated.

17. The method according to claim 12, wherein the receptor is non glycosylated.

18. The method according to claim 12, wherein the receptor is FcγRIIB/C(CD32) or FcγRIIIA/B (CD16b).

19. The method according to claim 12, wherein the amount administered to said patient in a single dose is between 1 and 20 mg/kg.

20. The method according to claim 12, wherein the receptor comprises a sequence selected from the group of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12 and SEQ ID NO: 13.

21. The method according to claim 12, wherein the FcγR is a recombinant non-glycosylated human soluble FcγRIIb preferably selected from the group of SEQ ID NO: 5, SEQ ID NO: 6, SEQ ID NO: 7 and SEQ ID NO: 8.


SEQUENCE LISTING (26 sequences)

SEQ ID NO: 1 - protein, 374 aa, Homo sapiens
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRKELKRKKKWDLEISLDSGHEKKVISSLQEDRHLEEELKCQEQKEEQLQEGVHRKEPQGAT

SEQ ID NO: 2 - protein, 291 aa, Homo sapiens
MGILSFLPVLATESDWADCKSPQPWGHMLLWTAVLFLAPVAGTPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISANPTNPDEADKVGAENTITYSLLMHPDALEEPDDQNRI

SEQ ID NO: 3 - protein, 280 aa, Homo sapiens
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFMEGEPLALRCHAWKDKLVYNVLYYRNGKAFKFFHWNSNLTILKTNISHNGTYHCSGMGKHRYTSAGISQYTVKGLQLPTPVWFHVLFYLAVGIMFLVNTVLWVTIRKELKRKKKWNLEISLDSGHEKKVISSLQEDRHLEEELKCQEQKEEQLQEGVHRKEPQGAT

SEQ ID NO: 4 - protein, 316 aa, Homo sapiens
MTMETQMSQNVCPRNLWLLQPLTVLLLLASADSQAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMGIIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 5 - protein, 310 aa, Homo sapiens
MGILSFLPVLATESDWADCKSPQPWGHMLLWTAVLFLAPVAGTPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISALPGYPECREMGETLPEKPANPTNPDEADKVGAENTITYSLLMHPDALEEPDDQNRI

SEQ ID NO: 6 - protein, 290 aa, Homo sapiens
MGILSFLPVLATESDWADCKSPQPWGHMLLWTAVLFLAPVAGTPAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISANPTNPDEADKVGAENTITYSLLMHPDALEEPDDQNRI

SEQ ID NO: 7 - protein, 291 aa, Homo sapiens; identical to SEQ ID NO: 2

SEQ ID NO: 8 - protein, 309 aa, Homo sapiens
MGILSFLPVLATESDWADCKSPQPWGHMLLWTAVLFLAPVAGTPAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISALPGYPECREMGETLPEKPANPTNPDEADKVGAENTITYSLLMHPDALEEPDDQNRI

SEQ ID NO: 9 - protein, 323 aa, Homo sapiens
MGILSFLPVLATESDWADCKSPQPWGHMLLWTAVLFLAPVAGTPAAPPKAVLKLEPQWINVLQEDSVTLTCRGTHSPESDSIPWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISANSTDPVKAAQFEPPGRQMIAIRKRQPEETNNDYETADGGYMTLNPRAPTDDDKNIYLTLPPNDHVNSNN

SEQ ID NO: 10 - protein, 276 aa, Homo sapiens
MGILSFLPVLATESDWADCKSPQPWGHMLLWTAVLFLAPVAGTPAAPPKAVLKLEPQWINVLQEDSVTLTCRGTHSPESDSIPWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISANSTDPVKAAQFEMLSCTHLDVK

SEQ ID NO: 11 - protein, 267 aa, Homo sapiens
MGILSFLPVLATESDWADCKSPQPWGHMLLWTAVLFLAPVAGTPAAPPKAVLKLEPQWINVLQEDSVTLTCRGTHSPESDSIPWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISATWTSNDCHQKETT

SEQ ID NO: 12 - protein, 254 aa, Homo sapiens [sequence omitted]

SEQ ID NO: 13 - protein, 85 aa, Homo sapiens
DLPKAVVFLEPQWYSVLEKDSVTLKCQGAYSPEDNSTQWFHNESLISSQASSYFIDAATVNDSGEYRCQTNLSTLSDPVQLEVHI

SEQ ID NO: 14 - DNA, 1125 bp, Homo sapiens
atgtggttcttgacaactctgctcctttgggttccagttgatgggcaagtggacaccacaaaggcagtgatcactttgcagcctccatgggtcagcgtgttccaagaggaaaccgtaaccttgcactgtgaggtgctccatctgcctgggagcagctctacacagtggtttctcaatggcacagccactcagacctcgacccccagctacagaatcacctctgccagtgtcaatgacagtggtgaatacaggtgccagagaggtctctcagggcgaagtgaccccatacagctggaaatccacagaggctggctactactgcaggtctccagcagagtcttcacggaaggagaacctctggccttgaggtgtcatgcgtggaaggataagctggtgtacaatgtgctttactatcgaaatggcaaagcctttaagtttttccactggaattctaacctcaccattctgaaaaccaacataagtcacaatggcacctaccattgctcaggcatgggaaagcatcgctacacatcagcaggaatatctgtcactgtgaaagagctatttccagctccagtgctgaatgcatctgtgacatccccactcctggaggggaatctggtcaccctgagctgtgaaacaaagttgctcttgcagaggcctggtttgcagctttacttctccttctacatgggcagcaagaccctgcgaggcaggaacacatcctctgaataccaaatactaactgctagaagagaagactctgggttatactggtgcgaggctgccacagaggatggaaatgtccttaagcgcagccctgagttggagcttcaagtgcttggcctccagttaccaactcctgtctggtttcatgtccttttctatctggcagtgggaataatgtttttagtgaacactgttctctgggtgacaatacgtaaagaactgaaaagaaagaaaaagtgggatttagaaatctctttggattctggtcatgagaagaaggtaatttccagccttcaagaagacagacatttagaagaagagctgaaatgtcaggaacaaaaagaagaacagctgcaggaaggggtgcaccggaaggagccccagggggccacgtag

SEQ ID NO: 15 - DNA, 567 bp, Homo sapiens
atgtggttcttgacaactctgctcctttggggctggctactactgcaggtctccagcagagtcttcatggaaggagaacctctggccttgaggtgtcatgcgtggaaggataagctggtgtacaatgtgctttactatcgaaatggcaaagcctttaagtttttccactggaattctaacctcaccattctgaaaaccaacataagtcacaatggcacctaccattgctcaggcatgggaaagcatcgctacacatcagcaggaatatcacaatacactgtgaaaggcctccagttaccaactcctgtctggtttcatgtccttttctatctggcagtgggaataatgtttttagtgaacactgttctctgggtgacaatacgtaaagaactgaaaagaaagaaaaagtggaatttagaaatctctttggattctggtcatgagaagaaggtaatttccagccttcaagaagacagacatttagaagaagagctgaaatgtcaggaacaaaaagaagaacagctgcaggaaggggtgcaccggaaggagccccagggggccacgtag

SEQ ID NO: 16 - DNA, 843 bp, Homo sapiens
atgtggttcttgacaactctgctcctttgggttccagttgatgggcaagtggacaccacaaaggcagtgatcactttgcagcctccatgggtcagcgtgttccaagaggaaaccgtaaccttgcactgtgaggtgctccatctgcctgggagcagctccacacagtggtttctcaatggcacagccactcagacctcgacccccagctacagaatcacctctgccagtgtcaatgacagtggtgaatacaggtgccagagaggtctctcagggcgaagtgaccccatacagctggaaatccacagaggctggctactactgcaggtctccagcagagtcttcatggaaggagaacctctggccttgaggtgtcatgcgtggaaggataagctggtgtacaatgtgctttactatcgaaatggcaaagcctttaagtttttccactggaattctaacctcaccattctgaaaaccaacataagtcacaatggcacctaccattgctcaggcatgggaaagcatcgctacacatcagcaggaatatcacaatacactgtgaaaggcctccagttaccaactcctgtctggtttcatgtccttttctatctggcagtgggaataatgtttttagtgaacactgttctctgggtgacaatacgtaaagaactgaaaagaaagaaaaagtggaatttagaaatctctttggattctggtcatgagaagaaggtaatttccagccttcaagaagacagacatttagaagaagagctgaaatgtcaggaacaaaaagaagaacagctgcaggaaggggtgcaccggaaggagccccagggggccacgtag

SEQ ID NO: 17 - DNA, 951 bp, Homo sapiens
atgactatggagacccaaatgtctcagaatgtatgtcccagaaacctgtggctgcttcaaccattgacagttttgctgctgctggcttctgcagacagtcaagctgctcccccaaaggctgtgctgaaacttgagcccccgtggatcaacgtgctccaggaggactctgtgactctgacatgccagggggctcgcagccctgagagcgactccattcagtggttccacaatgggaatctcattcccacccacacgcagcccagctacaggttcaaggccaacaacaatgacagcggggagtacacgtgccagactggccagaccagcctcagcgaccctgtgcatctgactgtgctttccgaatggctggtgctccagacccctcacctggagttccaggagggagaaaccatcatgctgaggtgccacagctggaaggacaagcctctggtcaaggtcacattcttccagaatggaaaatcccagaaattctcccatttggatcccaccttctccatcccacaagcaaaccacagtcacagtggtgattaccactgcacaggaaacataggctacacgctgttctcatccaagcctgtgaccatcactgtccaagtgcccagcatgggcagctcttcaccaatggggatcattgtggctgtggtcattgcgactgctgtagcagccattgttgctgctgtagtggccttgatctactgcaggaaaaagcggatttcagccaattccactgatcctgtgaaggctgcccaatttgagccacctggacgtcaaatgattgccatcagaaagagacaacttgaagaaaccaacaatgactatgaaacagctgacggcggctacatgactctgaaccccagggcacctactgacgatgataaaaacatctacctgactcttcctcccaacgaccatgtcaacagtaataactaa

SEQ ID NO: 18 - DNA, 933 bp, Homo sapiens
atgggaatcctgtcattcttacctgtccttgccactgagagtgactgggctgactgcaagtccccccagccttggggtcatatgcttctgtggacagctgtgctattcctggctcctgttgctgggacacctgcagctcccccaaaggctgtgctgaaactcgagccccagtggatcaacgtgctccaggaggactctgtgactctgacatgccgggggactcacagccctgagagcgactccattcagtggttccacaatgggaatctcattcccacccacacgcagcccagctacaggttcaaggccaacaacaatgacagcggggagtacacgtgccagactggccagaccagcctcagcgaccctgtgcatctgactgtgctttctgagtggctggtgctccagacccctcacctggagttccaggagggagaaaccatcgtgctgaggtgccacagctggaaggacaagcctctggtcaaggtcacattcttccagaatggaaaatccaagaaattttcccgttcggatcccaacttctccatcccacaagcaaaccacagtcacagtggtgattaccactgcacaggaaacataggctacacgctgtactcatccaagcctgtgaccatcactgtccaagctcccagctcttcaccgatggggatcattgtggctgtggtcactgggattgctgtagcggccattgttgctgctgtagtggccttgatctactgcaggaaaaagcggatttcagctctcccaggataccctgagtgcagggaaatgggagagaccctccctgagaaaccagccaatcccactaatcctgatgaggctgacaaagttggggctgagaacacaatcacctattcacttctcatgcacccggatgctctggaagagcctgatgaccagaaccgtatttag

SEQ ID NO: 19 - DNA, 798 bp, Homo sapiens
tctagcgaggtgacagcgtagaaccagagaatttgtttgccctctagggtagaatccgccaagctttgagagaaggctgtgactgctgtgctctgggcgccagctcgctccagggagtggtgggaatcctgtcattcttacctgtccttgccactgagagtgactgggctgactgcaagtccccccagccttggggtcatatgcttctgtggacagctgtgctattcctggctcctgttgctgggacacctgcagctcccccaaaggctgtgctgaaactcgagccccagtggatcaacgtgctccaggaggactctgtgactctgacatgccgggggactcacagccctgagagcgactccattcagtggttccacaatgggaatctcattcccacccacacgcagcccagctacaggttcaaggccaacaacaatgacagcggggagtacacgtgccagactggccagaccagcctcagcgaccctgtgcatctgactgtgctttctgagtggctggtgctccagacccctcacctggagttccaggagggagaaaccatcgtgctgaggtgccacagctggaaagacaagcctctggtcaaggtcacattcttccagaatggaaaatccaagaaattttcccgttcggatcccaacttctccatcccacaagcaaaccacagtcccagtgggtgattaccactgcacaggaaaaaatagggctacaccctgtactcatccaagccctgtggaccatcactgttcaaagctccccaacttcttcacggatggggga

SEQ ID NO: 20 - DNA, 876 bp, Homo sapiens
atgggaatcctgtcattcttacctgtccttgccactgagagtgactgggctgactgcaagtccccccagccttggggtcatatgcttctgtggacagctgtgctattcctggctcctgttgctgggacacctgcagctcccccaaaggctgtgctgaaactcgagccccagtggatcaacgtgctccaggaggactctgtgactctgacatgccgggggactcacagccctgagagcgactccattcagtggttccacaatgggaatctcattcccacccacacgcagcccagctacaggttcaaggccaacaacaatgacagcggggagtacacgtgccagactggccagaccagcctcagcgaccctgtgcatctgactgtgctttctgagtggctggtgctccagacccctcacctggagttccaggagggagaaaccatcgtgctgaggtgccacagctggaaggacaagcctctggtcaaggtcacattcttccagaatggaaaatccaagaaattttcccgttcggatcccaacttctccatcccacaagcaaaccacagtcacagtggtgattaccactgcacaggaaacataggctacacgctgtactcatccaagcctgtgaccatcactgtccaagctcccagctcttcaccgatggggatcattgtggctgtggtcactgggattgctgtagcggccattgttgctgctgtagtggccttgatctactgcaggaaaaagcggatttcagccaatcccactaatcctgatgaggctgacaaagttggggctgagaacacaatcacctattcacttctcatgcacccggatgctctggaagagcctgatgaccagaaccgtatttag

SEQ ID NO: 21 - DNA, 930 bp, Homo sapiens
atgggaatcctgtcattcttacctgtccttgccactgagagtgactgggctgactgcaagtccccccagccttggggtcatatgcttctgtggacagctgtgctattcctggctcctgttgctgggacacctgctcccccaaaggctgtgctgaaactcgagccccagtggatcaacgtgctccaggaggactctgtgactctgacatgccgggggactcacagccctgagagcgactccattcagtggttccacaatgggaatctcattcccacccacacgcagcccagctacaggttcaaggccaacaacaatgacagcggggagtacacgtgccagactggccagaccagcctcagcgaccctgtgcatctgactgtgctttctgagtggctggtgctccagacccctcacctggagttccaggagggagaaaccatcgtgctgaggtgccacagctggaaggacaagcctctggtcaaggtcacattcttccagaatggaaaatccaagaaattttcccgttcggatcccaacttctccatcccacaagcaaaccacagtcacagtggtgattaccactgcacaggaaacataggctacacgctgtactcatccaagcctgtgaccatcactgtccaagctcccagctcttcaccgatggggatcattgtggctgtggtcactgggattgctgtagcggccattgttgctgctgtagtggccttgatctactgcaggaaaaagcggatttcagctctcccaggataccctgagtgcagggaaatgggagagaccctccctgagaaaccagccaatcccactaatcctgatgaggctgacaaagttggggctgagaacacaatcacctattcacttctcatgcacccggatgctctggaagagcctgatgaccagaaccgtatttag

SEQ ID NO: 22 - DNA, 972 bp, Homo sapiens
atgggaatcctgtcattcttacctgtccttgccactgagagtgactgggctgactgcaagtccccccagccttggggtcatatgcttctgtggacagctgtgctattcctggctcctgttgctgggacacctgcagctcccccaaaggctgtgctgaaactcgagccccagtggatcaacgtgctccaagaggactctgtgactctgacatgccgggggactcacagccctgagagcgactccattccgtggttccacaatgggaatctcattcccacccacacgcagcccagctacaggttcaaggccaacaacaatgacagcggggagtacacgtgccagactggccagaccagcctcagcgaccctgtgcatctgactgtgctttctgagtggctggtgctccagacccctcacctggagttccaggagggagaaaccatcgtgctgaggtgccacagctggaaggacaagcctctggtcaaggtcacattcttccagaatggaaaatccaagaaattttcccgttcggatcccaacttctccatcccacaagcaaaccacagtcacagtggtgattaccactgcacaggaaacataggctacacgctgtactcatccaagcctgtgaccatcactgtccaagctcccagctcttcaccgatggggatcattgtggctgtggtcactgggattgctgtagcggccattgttgctgctgtagtggccttgatctactgcaggaaaaagcggatttcagccaattccactgatcctgtgaaggctgcccaatttgagccacctggacgtcaaatgattgccatcagaaagagacaacctgaagaaaccaacaatgactatgaaacagctgacggcggctacatgactctgaaccccagggcacctactgacgatgataaaaacatctacctgactcttcctcccaacgaccatgtcaacagtaataactaa

SEQ ID NO: 23 - DNA, 831 bp, Homo sapiens
atgggaatcctgtcattcttacctgtccttgccactgagagtgactgggctgactgcaagtccccccagccttggggtcatatgcttctgtggacagctgtgctattcctggctcctgttgctgggacacctgcagctcccccaaaggctgtgctgaaactcgagccccagtggatcaacgtgctccaagaggactctgtgactctgacatgccgggggactcacagccctgagagcgactccattccgtggttccacaatgggaatctcattcccacccacacgcagcccagctacaggttcaaggccaacaacaatgacagcggggagtacacgtgccagactggccagaccagcctcagcgaccctgtgcatctgactgtgctttctgagtggctggtgctccagacccctcacctggagttccaggagggagaaaccatcgtgctgaggtgccacagctggaaggacaagcctctggtcaaggtcacattcttccagaatggaaaatccaagaaattttcccgttcggatcccaacttctccatcccacaagcaaaccacagtcacagtggtgattaccactgcacaggaaacataggctacacgctgtactcatccaagcctgtgaccatcactgtccaagctcccagctcttcaccgatggggatcattgtggctgtggtcactgggattgctgtagcggccattgttgctgctgtagtggccttgatctactgcaggaaaaagcggatttcagccaattccactgatcctgtgaaggctgcccaatttgagatgctttcctgcacccacctggacgtcaaatga

SEQ ID NO: 24 - DNA, 804 bp, Homo sapiens
atgggaatcctgtcattcttacctgtccttgccactgagagtgactgggctgactgcaagtccccccagccttggggtcatatgcttctgtggacagctgtgctattcctggctcctgttgctgggacacctgcagctcccccaaaggctgtgctgaaactcgagccccagtggatcaacgtgctccaagaggactctgtgactctgacatgccgggggactcacagccctgagagcgactccattccgtggttccacaatgggaatctcattcccacccacacgcagcccagctacaggttcaaggccaacaacaatgacagcggggagtacacgtgccagactggccagaccagcctcagcgaccctgtgcatctgactgtgctttctgagtggctggtgctccagacccctcacctggagttccaggagggagaaaccatcgtgctgaggtgccacagctggaaggacaagcctctggtcaaggtcacattcttccagaatggaaaatccaagaaattttcccgttcggatcccaacttctccatcccacaagcaaaccacagtcacagtggtgattaccactgcacaggaaacataggctacacgctgtactcatccaagcctgtgaccatcactgtccaagctcccagctcttcaccgatggggatcattgtggctgtggtcactgggattgctgtagcggccattgttgctgctgtagtggccttgatctactgcaggaaaaagcggatttcagccacctggacgtcaaatgattgccatcagaaagagacaacctga

SEQ ID NO: 25 - DNA, 873 bp, Homo sapiens
atgggtggaggggctggggaaaggctgtttacttcctcctgtctagtcggtttggtccctttagggctccggatatctttggtgacttgtccactccagtgtggcatcatgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggctttttgggagtaaaaatgtgtcttcagagactgtgaacatcaccatcactcaaggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatga

SEQ ID NO: 26 - DNA, 702 bp, Homo sapiens
atgtggcagctgctcctcccaactgctctgctacttctagtttcagctggcatgcggactgaagatctcccaaaggctgtggtgttcctggagcctcaatggtacagcgtgcttgagaaggacagtgtgactctgaagtgccagggagcctactcccctgaggacaattccacacagtggtttcacaatgagaacctcatctcaagccaggcctcgagctacttcattgacgctgccacagtcaacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaagacaggaagtattttcatcataattctgacttccacattccaaaagccacactcaaagatagcggctcctacttctgcagggggcttgttgggagtaaaaatgtgtcttcagagactgtgaacatcaccatcactcaaggtttggcagtgtcaaccatctcatcattctctccacctgggtaccaagtctctttctgcttggtgatggtactcctttttgcagtggacacaggactatatttctctgtgaagacaaacatttga